(19) United States
(12) Patent Application Publication
(10) Pub. No.: US 2023/0111342 A1
(43) Pub. Date: Apr. 13, 2023

(54) COPPER NANOCATALYST, METHOD FOR PREPARING THE SAME, AND APPLICATION OF THE SAME IN THE SYNTHESIS OF ACETATE OR AMMONIA

(71) Applicant: University of Electronic Science and Technology of China, Chengdu (CN)

(72) Inventors: Yijin KANG, Chengdu (CN); Xianbiao FU, Chengdu (CN)

(73) Assignee: University of Electronic Science and Technology of China, Chengdu (CN)

(21) Appl. No.: 18/080,718

(22) Filed: Dec. 13, 2022

Related U.S. Application Data
(63) Continuation of application No. 16/892,295, filed on Jun. 4, 2020.

(30) Foreign Application Priority Data
Jun. 4, 2019 (CN) 201910482607.0

(51) Int. Cl. C25B 11/081; C25B 1/00; C25B 3/25; C25B 11/051

(52) U.S. Cl. CPC C25B 11/081; C25B 1/00; C25B 3/25; C25B 11/051

(57) ABSTRACT

A copper nanocatalyst, a method for preparing the copper nanocatalyst, and an application of the copper nanocatalyst in the synthesis of acetate or ammonia are provided. The copper nanocatalyst includes a substrate and an active agent loaded on the substrate. The method includes: preparing a cleaning agent by using an ethanol and a deionized; immersing the active agent in the cleaning agent, ultrasonically cleaning for 5-10 min at a frequency of 4×10⁴ Hz-8×10⁴ Hz, and drying for later use; mixing the cleaned active agent and a conductive binder according to a mass ratio of 1:19-9:1 of the active agent to the conductive binder, adding the ethanol, and fully stirring and dispersing to obtain a slurry; coating the slurry on a surface of the carbon paper, and drying the carbon paper by blowing through nitrogen flow to obtain the catalyst.

### CROSS REFERENCE TO THE RELATED APPLICATIONS

This application is a continuation application of U.S. application Ser. No. 16/892,295, which is based upon and claims priority to Chinese Patent Application No. 201910482607.0, filed on Jun. 4, 2019, the entire contents of which are incorporated herein by reference.

### TECHNICAL FIELD

The present invention belongs to the technical field of catalysts, and particularly relates to a copper nanocatalyst, a method for preparing the same, and an application of the same in the synthesis of acetate or ammonia.

### BACKGROUND

On the premise of limited reserves of fossil energy, efforts are being made globally to find new energy, and the most promising hydrogen energy system is hydrogen as an energy carrier. However, the hydrogen energy system cannot provide chemical products other than energy sources for human society like the petroleum energy system. Scientists have focused on synthesizing high-value multi-carbon compounds starting from small molecules (e.g., hydrogen, oxygen, water, carbon monoxide, carbon dioxide) that are available in large quantities in the environment, thereby meeting the demand of daily chemical products. However, the synthesis route has the problems of low reaction rate, high difficulty in generating high-value products, and high industrial production cost caused by poor product selectivity. Therefore, inventing high-efficiency and high-selectivity catalysts is the main target for upgrading the chemical industry under the hydrogen energy system.

Ammonia is not only an essential feedstock chemical for the manufacture of fertilizers, pharmaceuticals, inorganic and organic nitrogen compounds, but also an ideal carbon-free fuel, containing 17.5 wt % hydrogen. Most of the ammonia synthesis in the world is implemented via the Haber-Bosch process, which consumes 1-2% of the annual global energy supply and generates 1% of carbon dioxide (CO₂) in the world, because the process requires substantial driving force (typically 500° C. and 200 atm) and hydrogen gas (H₂). The substantial driving force is obtained from the high energy consumption, which is excessively dependent on fossil fuels and is responsible for about half of CO₂ emissions. Meanwhile H₂ is produced by coal gasification, and the process thereof accounts the other half of CO₂ emissions in the entire process. Moreover, a substantial amount of ammonia that is released to the environment is eventually oxidized to nitrate via nitrification (NH₄⁺→NO₂⁻→NO₃⁻), causing an unbalanced nitrogen cycle and nitrate pollution. Therefore, it is imperative to develop an efficient and clean ammonia synthesis process for mitigating environmental concerns.

Having broken through the chemical thermodynamic limitations of the Haber-Bosch process, the electrochemical ammonia synthesis can be carried out under ambient conditions, which is beneficial to reduce energy consumption and relieve the problem of excessive emission of carbon dioxide. Moreover, the electrochemical ammonia synthesis takes water as a proton source to circumvent environmental pollution in the hydrogen production process. Recently, tremendous efforts have been made to improve the performance of electrochemical nitrogen reduction reaction (NRR) to ammonia with water as a proton source under ambient conditions. However, extremely low ammonia yield rate and current efficiency (typically 0.1-30 μg mg⁻¹_{cat} h⁻¹ and 0.1-10%, respectively) limit the potential application of direct electroreduction of nitrogen. The substantially low water solubility of nitrogen is the root of the problem, manifested in the Henry's Law constant of K_{H}=6.24×10⁻⁴ mol L⁻¹atm⁻¹. Seeking and activating the water-soluble and accessible nitrogenous species in nitrogen cycling is a great challenge for efficient electrochemical ammonia synthesis.

### SUMMARY

An objective of the present invention is to provide a copper nanocatalyst and a method for preparing the same, which avoids the high energy consumption and high pollution of the Haber-Bosch process for ammonia synthesis and the low efficiency of ammonia production via electrochemical reduction of nitrogen.

To achieve the objective, the present invention adopts the following technical solution. A catalyst includes a substrate and an active agent loaded on the substrate, wherein a loading amount of the active agent on the substrate is 0.1-3.0 mg/cm², and the active agent is a copper nanomaterial with an exposed 50%-99% (111) crystal face.

Based on the above-mentioned technical solution, the present invention can be further improved as follows.

Further, the substrate comprises a carbon paper, a carbon cloth, a silicon oxide film, or an aluminum oxide film.

Further, the active agent is a copper nanosheet, a copper nanopolyhedron or a copper nanowire with an exposed (111) crystal face.

Further, the copper nanopolyhedron is at least one of a copper regular nanotetrahedron, a copper nanocube, a copper regular nanooctahedron and a copper regular nanoicosahedron.

Further, the loading amount of the active agent on the substrate is 1.0 mg/cm².

A method for preparing the catalyst of the present invention includes the following steps:

(1) preparing a cleaning agent by using ethanol and deionized water, wherein a volume ratio of the ethanol to the deionized water in the cleaning agent is 5-90:10-95; immersing the active agent in the cleaning agent, ultrasonically cleaning for 5-10 min at a frequency of 4×10⁴ Hz-8×10⁴ Hz, and drying for later use;

(2) mixing the cleaned active agent and a conductive binder according to a mass ratio of 1:19-9:1, adding ethanol, and fully stirring and dispersing to obtain slurry; and

(3) coating the slurry on the surface of the carbon paper and drying the carbon paper by blowing through nitrogen flow to obtain the catalyst.

The method for preparing the active agent used in the step (1) includes the following steps: dissolving copper nitrate, ascorbic acid, hexamethylenetetramine and hexadecyltrimethylammonium bromide in the deionized water according to a molar ratio of 1:0.1-0.5:0.1-0.5:0.5-1, stirring to form a homogeneous solution, placing the homogeneous solution in an oil bath at 70-100° C. to react for 1-5 h, cooling, washing with a mixed solution of the ethanol and water, centrifuging, taking a precipitate, and drying to obtain the active agent.

The conductive binder used in the step (2) is Nafion, and a mass ratio of the Nafion to the active agent is 4:1.

The catalyst of the invention has about 48% selectivity during catalytic conversion of carbon monoxide or carbon dioxide to acetate (salt), while during catalytic conversion of nitric acid (salt) to ammonia, the yield and selectivity are close to 100%. Therefore, the catalyst in the invention can be used as a high efficient catalyst for the synthesis of acetate or ammonia.

The present invention has the advantages as follows. The catalyst of the present invention has regular morphology, copper (111) basal plane of the nanosheet, well-defined structure, low cost, and high efficiency and selectivity of electroreduction of nitrate to ammonia. The catalyst can efficiently convert nitrate into ammonia at ambient temperature and pressure, which not only breaks through mass transfer barriers in the process of electroreduction of nitrogen to ammonia but also reduces energy consumption and relieves the problem of excessive emission of carbon dioxide during the Haber-Bosch process.

### DETAILED DESCRIPTION OF THE DRAWINGS

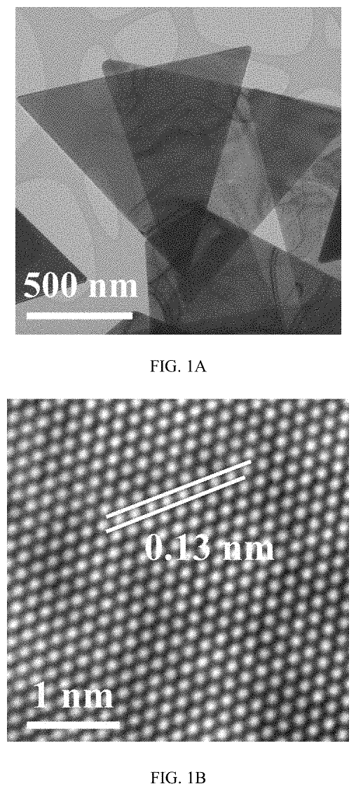

FIGS. 1A-1C show a structural characterization of a copper nanosheet;

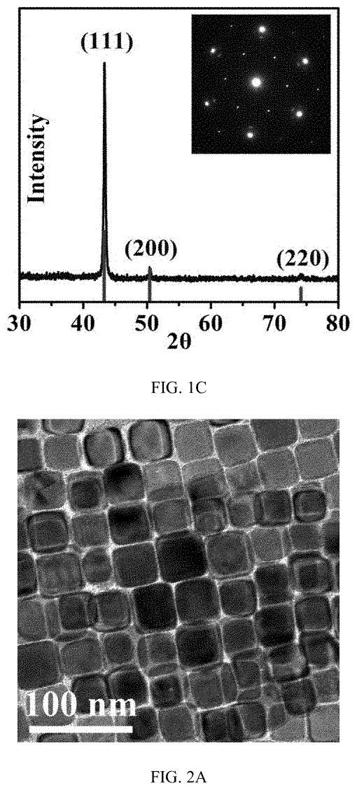

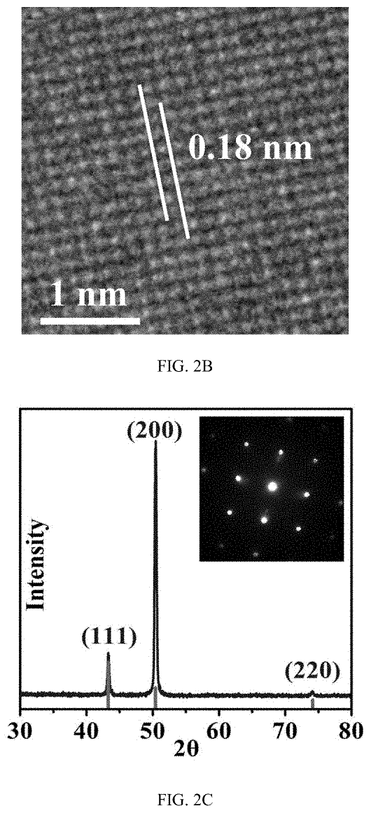

FIGS. 2A-2C show a structural representation of a copper nanocube;

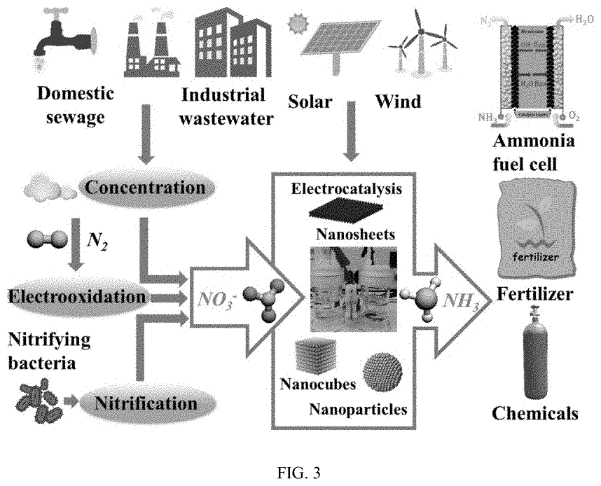

FIG. 3 is a schematic diagram showing a route for ammonia synthesis via electroreduction of nitrate; and

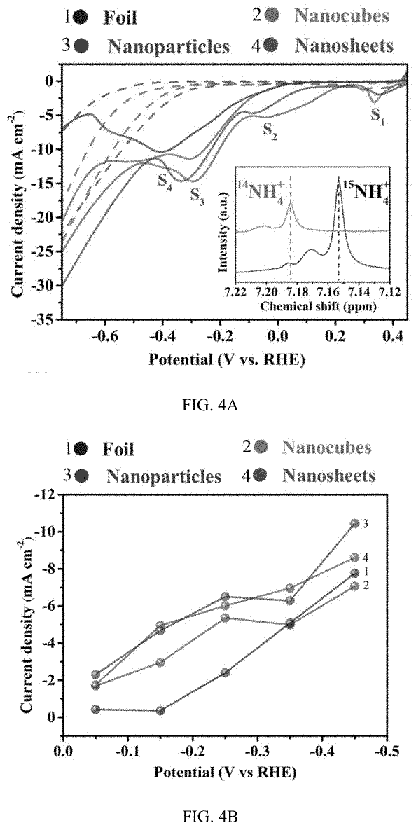

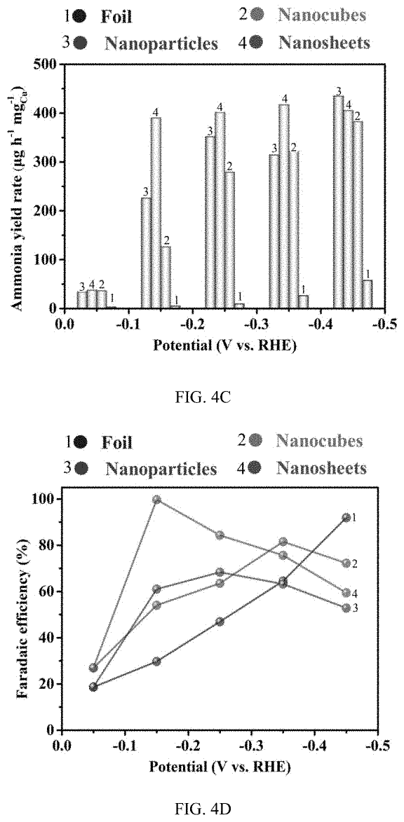

FIGS. 4A-4D show the results of the electroreduction of nitrate to ammonia.

### DETAILED DESCRIPTION OF THE EMBODIMENTS

The present invention is described in detail below in conjunction with the embodiments.

### Embodiment 1

A copper nanocatalyst includes a carbon paper substrate and a copper nanosheet loaded on the carbon paper, wherein the loading capacity of the copper nanosheet on the carbon paper is about 1.0 mg/cm². The method for preparing the catalyst is as follows.

(1) Synthesis of copper nanosheet: the copper nitrate, ascorbic acid, hexamethylenetetramine and hexadecyltrimethylammonium bromide are dissolved in deionized water according to the molar ratio of 1:0.1:0.5:0.5, and are stirred to form a homogeneous solution. The solution is placed in an oil bath at 100° C. to react for 2 hours, and is then cooled. The mixed solution of ethanol and water is added to the solution for washing and centrifuging, and a precipitate is taken to dry to obtain an active agent, wherein the active agent is the copper nanosheet.

(2) Cleaning of copper nanosheet: the ethanol and the deionized water are adopted to prepare a cleaning agent, wherein the volume ratio of the ethanol to the deionized water in the prepared cleaning agent is 1:9. The active agent is immersed in the cleaning agent and is ultrasonically cleaned for 8 min at a frequency of 6×10⁴ Hz, and is then dried for later use.

(3) Preparation of slurry: the Nafion conductive binder with a concentration of 10% is added into the cleaned active agent, wherein the mass ratio of the added Nafion to the active agent is 4:1, and then a proper amount of ethanol is added, and after fully stirring and dispersing, the slurry is obtained.

(4) Preparation of catalyst: the slurry is uniformly coated on the surface of the carbon paper, and is then dried by blowing through nitrogen flow to obtain the catalyst.

### Embodiment 2

A copper nanocatalyst includes a carbon cloth substrate and a copper nanocube loaded on the carbon cloth, wherein the loading capacity of the copper nanocube on the carbon cloth is about 3.0 mg/cm². The method for preparing the catalyst is as follows.

(1) Cleaning of copper nanocube: the ethanol and the deionized water are adopted to prepare a cleaning agent, wherein the volume ratio of the ethanol to the deionized water in the prepared cleaning agent is 1:1. The prepared copper nanocube is then immersed in the cleaning agent and is ultrasonically cleaned for 5 min at a frequency of 8×10⁴ Hz, and is then dried for later use.

(2) Preparation of slurry: the Nafion conductive binder with a concentration of 10% is added into the cleaned active agent, wherein the mass ratio of the added Nafion to the active agent is 1:1, and then a proper amount of ethanol is added, and after fully stirring and dispersing, the slurry is obtained.

(3) Preparation of catalyst: the slurry is uniformly coated on the surface of the carbon cloth, and is then dried by blowing through nitrogen flow to obtain the catalyst.

### Embodiment 3

A copper nanocatalyst includes a carbon paper substrate and a copper nanowire loaded on the carbon paper, wherein the loading amount of the copper nanowire on the carbon paper is about 0.5 mg/cm². The method for preparing the catalyst is as follows.

(1) Cleaning of copper nanowire: the ethanol and the deionized water are adopted to prepare a cleaning agent, wherein the volume ratio of the ethanol to the deionized water in the prepared cleaning agent is 4:1. The prepared copper nanowire is immersed into the cleaning agent and is ultrasonically cleaned for 10 min at a frequency of 4×10⁴ Hz, and is then dried for later use.

(2) Preparation of slurry: the Nafion conductive binder with a concentration of 10% is added into the cleaned active agent, wherein the mass ratio of the added Nafion to the active agent is 1:4, and then a proper amount of ethanol is added, and after fully stirring and dispersing, the slurry is obtained.

(3) Preparation of catalyst: the slurry is uniformly coated on the surface of the carbon paper and is then dried by blowing through nitrogen flow to obtain the catalyst.

Analysis of Results

The copper nanosheet synthesized in Embodiment 1 was taken to analyze the structure thereof, and the result is shown in FIGS. 1A-1C, wherein, FIG. 1A represents transverse electric and magnetic field (TEM), FIG. 1B represents high resolution transmission electron microscopy (HRTEM) and FIG. 1C represents X-Ray Diffraction (XRD). The copper nanocube synthesized in Embodiment 2 was taken to analyze the structure thereof, and the result is shown in FIGS. 2A-2C, wherein, FIG. 2A represents TEM, FIG. 2B represents HRTEM, and FIG. 2C represents XRD. From FIGS. 1A-1C and FIGS. 2A-2C, it can be seen that the copper nanomaterials have regular morphology and a well-defined structure.

The catalyst prepared in the Embodiment 1 was adopted to test the electrochemical reduction of nitrate to ammonia, and the test path is shown in FIG. 3, wherein the test condition is ambient temperature and pressure, and the applied potential is from −0.1 to −1.0V (vs RHE). The test results are shown in FIGS. 4A-4D, wherein, FIG. 4A is electrochemical data, and the test conditions are as follows: 0.1M potassium hydroxide solution (dotted line), 0.1 M potassium hydroxide solution presence of 10 mM potassium nitrate solution (solid line), scanning speed 20 mA/s, and the inset is the ¹H nuclear magnetic resonance spectrogram calibrated by K¹⁵NO₃ (98 atom %¹⁵N); FIG. 4B is the current density. From FIG. 4A and FIG. 4B, it can be seen that nitrate can be converted to ammonia at lower potentials by the catalyst of the present invention, and the conversion rate increases as the current increases. And FIG. 4C is the synthesis rate of the ammonia; FIG. 4D is faradaic efficiency (i.e., yield). From FIG. 4C and FIG. 4D, it can be seen that at −0.15V versus RHE, the ammonia yield of the catalyst with the copper nanosheet as the active agent is 390.1 mug mg⁻¹_{Cu} h⁻¹, and is close to 100%, which shows that the catalyst of the present invention can efficiently convert nitrate into ammonia, and has the advantages of low energy consumption, no pollution, and meeting the requirements of the green chemical industry.

Although the embodiments of the present invention has been described in detail above, they should not be construed as a limitation to the scope of the present invention. Various modifications and variations made by those skilled in the art within the scope described in the claims without creative work shall fall within the scope of protection of the present invention.

What is claimed is:

1. A copper nanocatalyst for synthesizing ammonia from nitrate comprising a substrate and an active agent loaded on the substrate, wherein a loading amount of the active agent on the substrate is 0.1-3.0 mg/cm², and the active agent is a copper nanopolyhedron with an exposed 50%-99% (111) crystal face, wherein the copper nanopolyhedron is at least one selected from the group consisting of a copper regular nanotetrahedron, a copper regular nanooctahedron, a carbon nanocube, and a copper regular nanoicosahedron.

2. The copper nanocatalyst according to claim 1, wherein, the substrate comprises a carbon paper, a carbon cloth, a silicon oxide film, or an aluminum oxide film.

3. (canceled)

4. (canceled)

5. The copper nanocatalyst according to claim 1, wherein, the loading amount of the active agent on the substrate is 1.0 mg/cm².

6. A method for preparing the copper nanocatalyst according to claim 1, comprising the following steps:
(1) preparing a cleaning agent by using an ethanol and a deionized water, wherein a volume ratio of the ethanol to the deionized water in the cleaning agent is 5-90:10-95; immersing the active agent in the cleaning agent, ultrasonically cleaning the active agent for 5-10 min at a frequency of 4×10⁴ Hz-8×10⁴ Hz to obtain a cleaned active agent, and drying the cleaned active agent for later use;
(2) mixing the cleaned active agent and a conductive binder according to a mass ratio of 1:19-9:1 of the cleaned active agent to the conductive binder to obtain a mixture, adding the ethanol to the mixture to obtain a first solution, and fully stirring and dispersing the first solution to obtain a slurry; and
(3) coating the slurry on a surface of the substrate and drying the substrate by blowing through nitrogen flow to obtain the copper nanocatalyst, wherein an active agent of the copper nanocatalyst is a copper nanopolyhedron with an exposed 50%-99% (111) crystal face, the copper nanopolyhedron is at least one selected from the group consisting of a copper regular nanotetrahedron, a carbon nanocube, a copper regular nanooctahedron, and a copper regular nanoicosahedron, and a loading amount of the active agent on the substrate is 0.1-3.0 mg/cm².

7. The method according to claim 6, wherein, a method for preparing the active agent comprises the following steps: dissolving and stirring copper nitrate, ascorbic acid, hexamethylenetetramine and hexadecyltrimethylammonium bromide in the deionized water to form a homogeneous solution, placing the homogeneous solution in an oil bath at 70-100° C. to react for 1-5 h to obtain a second solution, cooling the second solution, washing the second solution with a mixed solution of the ethanol and water to obtain a third solution, centrifuging the third solution to obtain a precipitate, and drying the precipitate to obtain the active agent.

8. The method according to claim 7, wherein, a molar ratio of the copper nitrate, the ascorbic acid, the hexamethylenetetramine and the hexadecyltrimethylammonium bromide is 1:0.1-0.5:0.1-0.5:0.5-1.

9. The method according to claim 7, wherein, the conductive binder is Nafion, and a mass ratio of the Nafion to the active agent is 4:1.

10. A method of synthesizing acetate or ammonia, comprising:
contacting the copper nanocatalyst according to claim 1 with nitrate to synthesize ammonia.

11. The method according to claim 6, wherein, the substrate comprises a carbon paper, a carbon cloth, a silicon oxide film, or an aluminum oxide film.

12. The method according to claim 6, wherein, the active agent is a copper nanosheet, a copper nanopolyhedron or a copper nanowire, and the copper nanosheet, the copper nanopolyhedron or the copper nanowire has the exposed 50%-99% (111) crystal face.

13. The method according to claim 12, wherein, the copper nanopolyhedron is at least one selected the group consisting of a copper regular nanotetrahedron, a copper nanocube, a copper regular nanooctahedron and a copper regular nanoicosahedron.

14. The method according to claim 6, wherein, the loading amount of the active agent on the substrate is 1.0 mg/cm².

15. The method according to claim 10, wherein, the substrate comprises a carbon paper, a carbon cloth, a silicon oxide film, or an aluminum oxide film.

16. The method according to claim 10, wherein, the active agent is a copper nanosheet, a copper nanopolyhedron or a copper nanowire, and the copper nanosheet, the copper nanopolyhedron or the copper nanowire has the exposed 50%-99% (111) crystal face.

17. The method according to claim 16, wherein, the copper nanopolyhedron is at least one selected the group consisting of a copper regular nanotetrahedron, a copper nanocube, a copper regular nanooctahedron and a copper regular nanoicosahedron.

18. The method according to claim 10, wherein, the loading amount of the active agent on the substrate is 1.0 mg/cm².

19. The copper nanocatalyst according to claim 1, wherein the active agent is characterized by an x-ray diffraction pattern comprising a first peak between 40-45° 2θ, a second peak between 50-55° 2θ and a third peak between 70-75° 2θ.
